(19) United States
(12) Patent Application Publication
(10) Pub. No.: US 2019/0153382 A1
(43) Pub. Date: May 23, 2019

(54) INK-JET PRINTING OF TISSUES

(71) Applicant: WAKE FOREST UNIVERSITY HEALTH SCIENCES, Winston-Salem, NC (US)

(72) Inventors: James Yoo, Winston-Salem, NC (US); Tao Xu, Winston-Salem, NC (US); Anthony Atala, Winston-Salem, NC (US)

(21) Appl. No.: 16/227,690

(22) Filed: Dec. 20, 2018

Related U.S. Application Data
(63) Continuation of application No. 12/293,490, filed on Apr. 23, 2009, now abandoned.
(60) Provisional application No. 60/794,033, filed on Apr. 21, 2006.

(51) Int. Cl. C12N 5/00; B33Y 10/00; B29C 70/76

(52) U.S. Cl. CPC C12N 5/0062; B01L 3/0268; B29C 70/76; B33Y 10/00

(57) ABSTRACT

A method of forming an array of viable cells is carried out by ink-jet printing a cellular composition containing said cells on a substrate. At least two different types of viable mammalian cells are printed on the substrate, the at least two different types of viable mammalian cells selected to together form a tissue. In some embodiments at least three or four different viable mammalian cells are printed on the substrate, the cells selected to together form a tissue. In some embodiments one of the viable mammalian cell types is a stem cell. In some embodiments the method further comprises printing at least one support compound on the substrate, the support compound selected to form a tissue together with said cells. In some embodiments the method further comprises printing at least one growth factor on the substrate, the growth factor selected to cause the cells to form a tissue.

### FIELD OF THE INVENTION

The present invention concerns ink-jet printing of viable cells and arrays of cells so produced.

### BACKGROUND OF THE INVENTION

Living tissues maintain an inherent multi-cellular heterogeneous structure. Rebuilding of such complex structure requires subtle arrangements of different types of cells and extracellular matrices (ECM) at their specific anatomical target sites. To achieve tissue reconstitution, an effective method for a precise delivery of cells and biomaterials is needed. The inkjet printing technology has been applied to address this endeavor.

The following references are noted herein:

T. Boland et al., Ink jet printing of viable cells, U.S. Pat. No. 7,051,654;

W. Warren et al., Architecture tool and methods of use, U.S. Pat. No. 6,986,739; and

J. Barron et al., Biological laser printing via indirect photon-biomaterial interactions, US Patent Application Publication No. 2005/0018036.

### SUMMARY OF THE INVENTION

Although the capability of inkjet printing of viable single cells has been verified, the possibility of simultaneously printing multiple cell types to build viable heterogeneous cellular constructs has not been demonstrated to date. It has been found that distinct cell types can be mixed with support compounds (collagen gels) and printed into the target areas to form 3-dimensional tissue structures. Further, basic physiological functions and properties of each cell type within the structure can be maintained.

A first aspect of the invention is, in a method of forming an array of viable cells by ink-jet printing a cellular composition containing said cells on a substrate, the improvement comprising printing at least two different types of viable mammalian cells on said substrate, said at least two different types of viable mammalian cells selected to together form a tissue. In some embodiments at least three or four different viable mammalian cells are printed on said substrate, the cells selected to together form a tissue. In some embodiments one of said viable mammalian cell types is a stem cell. In some embodiments the method further comprises printing at least one support compound on said substrate, said support compound selected to form a tissue together with said cells. In some embodiments the method further comprises printing at least one growth factor on said substrate, the growth factor selected to cause the cells to form a tissue.

Example tissues, or tissue substitutes, that may be produced by the processes of the invention include nerve, skin, pancreatic islet, and bone tissue.

Since it is preferred to print three-dimensional arrays when forming tissues or tissue substitutes as described above, and since such printing may require substantially greater times than required in prior techniques, it is sometimes preferred to carry out the printing in a culture chamber or an environmentally controllable chamber to enhance the survival of cells after printing.

Another aspect of the invention is a method of forming an array of viable cells by ink-jet printing a cellular composition containing said cells on a substrate, the improvement comprising: printing viable stem cells (for example, amniotic fluid stem cells) on the substrate.

Another aspect of the invention is a method of forming an array of viable cells by ink jet printing a cellular composition containing said cells on a substrate, the improvement comprising: printing viable cancer cells on said substrate. Arrays produced by such methods are useful in screening compounds for efficacy in treating cancer by contacting the compound to the cancer cells. Arrays can be printed with normal or “control” cells adjacent to the cancer cells, so that the test compound may be concurrently contacted to the control cells, so that the differential effect of the test compound on cancer cells as compared to control cells may be evaluated.

The foregoing and other objects and aspects of the present invention are explained in greater detail in the drawings herein and the specification set forth below.

### BRIEF DESCRIPTION OF THE DRAWINGS

FIG. 1. (a) Multi-cellular “pie” structure. Morphologies of 3 distinct cell types in the “pie” structure: (b) SMC, (c) hAFSC, and (d) MS1. (e) Printed collagen scaffold with different color dyes.

FIG. 2. (a) Alizarin red staining of printed hAFSC within the 3-D collagen constructs after 25 days of culture. (b) I-V relationship of BSMC printed in the collagen constructs.

FIG. 3. (a) Fluorescent microscopic image of a 3-D “pie” before implantation. (b) Gross view of the retrieved “pie” 2 weeks post-implantation. (c) and (d) Fluorescent images of ECs and SMCs within the “pie” implant, respectively.

FIG. 4. (a) I-V relationship of printed SMCs before and 4 weeks after implantation. (b) MRI scanning of EC-printed constructs 8 weeks after implantation. (c) Micro-CT scanning of AFSC-printed samples 18 weeks after implantation. New engineered bone tissues were observed within the implants. (d) and (e) Immunohistochemical analyses of EC-printed constructs 8 weeks after implantation and AFSC-printed constructs 18 week after implantation, respectively.

### DETAILED DESCRIPTION OF THE PREFERRED EMBODIMENTS

“Support compound” as used herein may be any naturally occurring or synthetic support compound, including combinations thereof, suitable for the particular tissue or array being printed. In general the support compound is preferably physiologically acceptable or biocompatible. Suitable examples include but are not limited to alginate, collagen (including collagen VI), elastin, keratin, fibronectin, proteoglycans, glycoproteins, polylactide, polyethylene glycol, polycaprolactone, polycolide, polydioxanone, polyacrylates, polysulfones, peptide sequences, proteins and derivatives, oligopeptides, gelatin, elastin, fibrin, laminin, polymethacrylates, polyacetates, polyesters, polyamides, polycarbonates, polyanhydrides, polyamino acids carbohydrates, polysaccharides and modified polysaccharides, and derivatives and copolymers thereof (see, e.g., U.S. Pat. Nos. 6,991,652 and 6,969,480) as well as inorganic materials such as glass such as bioactive glass, ceramic, silica, alumina, calcite, hydroxyapatite, calcium phosphate, bone, and combinations of all of the foregoing.

“Growth factor” as used herein may be any naturally occurring or synthetic growth factor, including combinations thereof, suitable for the particular tissue or array being printed. Numerous growth factors are known to those skilled in the art. Examples include but are not limited to: insulin-like growth factor (e.g., IGF-1), transforming growth factor-beta (TGF-beta), bone-morphogenetic protein, fibroblast growth factor, platelet derived growth factor (PDGF), vascular-endothelial growth factor (VEGF), connective tissue growth factor (CTGF), basic fibroblast growth factor (bFGF), epidermal growth factor, fibroblast growth factor (FGF) (numbers 1, 2 and 3), osteopontin, bone morphogenetic protein-2, growth hormones such as somatotropin, cellular attractants and attachment agents, etc., and mixtures thereof. See, e.g., U.S. Pat. Nos. 7,019,192; 6,995,013; and 6,923,833.

“Cells” as used herein may be of any suitable species, and in some embodiments are of the same species as the subject into which tissues produced by the processes herein are implanted. Mammalian cells (including mouse, rat, dog, cat, monkey and human cells) are in some embodiments particularly preferred.

“Stem cell” as used herein refers to a cell that has the ability to replicate through numerous population doublings (e.g., at least 60-80), in some cases essentially indefinitely, and to differentiate into multiple cell types (e.g., is pluripotent or multipotent).

“Embryonic stem cell” as used herein refers to a cell that is derived from the inner cell mass of a blastocyst and that is pluripotent.

“Amniotic fluid stem cell” as used herein refers to a cell, or progeny of a cell, that (a) is found in, or is collected from, mammalian amniotic fluid, mammalian chorionic villus, and/or mammalian placental tissue, or any other suitable tissue or fluid from a mammalian donor, (b) is pluripotent; (c) has substantial proliferative potential, (d) optionally, but preferably, does not require feeder cell layers to grow in vitro, (e) optionally, but preferably, specifically binds c-kit antibodies (particularly at the time of collection, as the ability of the cells to bind c-kit antibodies may be lost over time as the cells are grown in vitro).

“Pluripotent” as used herein refers to a cell that has complete differentiation versatility, e.g., the capacity to grow into any of the animals cell types. A pluripotent cell can be self-renewing, and can remain dormant or quiescent with a tissue. Unlike a totipotent cell (e.g., a fertilized, diploid egg cell) a pluripotent cell cannot usually form a new blastocyst.

“Multipotent” as used herein refers to a cell that has the capacity to grow into any of a subset of the corresponding animals cell type. Unlike a pluripotent cell, a multipotent cell does not have the capacity to form all of the cell types of the corresponding animal.

“Cancer cells” as used herein may be of any type, including but not limited to leukemia, lymphoma, breast, lung, colon, prostate, ovarian, skin, melanoma, and brain cancer cells.

Subjects that may be implanted with constructs or arrays of the present invention include both human subjects and animal subjects (particularly mammalian subjects such as dogs, cats, horses, pigs, sheep, cows, etc.) for veterinary purposes.

The disclosures of all United States patent references cited herein are to be incorporated herein by reference in their entirety.

### A. Tissue Printing.

Methods and compositions for the ink-jet printing of viable cells are known and described in, for example, T. Boland et al., US Patent Application No. 2004/0237822 (Dec. 2, 2004) (Clemson University) and W. Wilson and T. Boland, The Anatomical Record Part A, 272A: 491-496 (2003). While the present invention is primarily concerned with ink-jet printing of cells, the cells may be printed by other means as well, such as the methods and compositions for forming three-dimensional structures by deposition of viable cells described in W. Warren et al., U.S. Pat. No. 6,986,739 (Sciperio Inc.).

Although not required, cells can typically be printed in the form of a “cell composition” that contains a liquid carrier for the cells. The cell composition can be in the form of a suspension, solution, or any suitable form. Examples of suitable liquid carriers include, but are not limited to, water, ionic buffer solutions (e.g., phosphate buffer solution, citrate buffer solution, etc.), liquid media (e.g., modified Eagle's medium (“MEM”), Hanks' Balanced Salts, etc.), and so forth. For instance, the use of a liquid carrier in the cell composition can ensure adequate hydration and minimize evaporation of the cells after printing. However, the probability of obtaining viable cells in any given printed drop also decreases with decreasing cell concentration. (T. Boland, US Patent Application Publication No. 20040237822 at para 48)

Various mechanisms may be employed to facilitate the survival of the cells during and/or after printing. Specifically, compounds may be utilized that “support” the printed cells by providing hydration, nutrients, and/or structural support. These compounds may be applied to the substrate using conventional techniques, such as manually, in a wash or bath, through vapor deposition (e.g., physical or chemical vapor deposition), etc. These compounds may also be combined with the cell composition before and/or during printing, or may be printed or otherwise applied to the substrate (e.g., coated) as a separate layer beneath, above, and/or between cell layers. For example, one such support compound is a gel having a viscosity that is low enough under the printing conditions to pass through the nozzle of the printer head, and that can gel to a stable shape during and/or after printing. Such viscosities are typically within the range of from about 0.5 to about 50 centipoise, in some embodiments from about 1 to about 20 centipoise, and in some embodiments, from about 1 to about 10 centipoise. Some examples of suitable gels that may be used in the present invention include, but are not limited to, agars, collagen, hydrogels, etc. One example of a collagen gel for facilitating cell growth is described in Collagen As a Substrate for Cell Growth and Differentiation, Methods in Enzymology, Strom and Michalopoulous, Vol. 82. 544-555 (1982) (T. Boland at para 50).

Besides gels, other support compounds may also be utilized in the present invention. Extracellular matrix analogs, for example, may be combined with support gels to optimize or functionalize the gel. One or more growth factors may also be introduced in the printed cell arrays. For example, slow release microspheres that contain one or more growth factors in various concentrations and sequences may be combined with the cell composition to accelerate and direct the cell fusion process. Other suitable support compounds might include those that aid in avoiding apoptosis and necrosis of the developing structures. For example, survival factors (e.g., basic fibroblast growth factor) may be added. In addition, transient genetic modifications of cells having antiapoptotic (e.g., bcl-2 and telomerase) and/or blocking pathways may be included in cell aggregates to be printed according to the invention. Adhesives may also be utilized to assist in the survival of the cells after printing. For instance, soft tissue adhesives, such a cyanoacrylate esters, fibrin sealant, and/or gelatin-resorcinol-formaldehyde glues, may be utilized to inhibit nascent constructs from being washed off or moved following printing of a layer. In addition, adhesives, such as arginine-glycine-aspartic acid ligands, may enhance the adhesion of cells to a gelling polymer or other support compound. In addition, extracellular proteins, extracellular protein analogs, etc., may also be utilized (T. Boland at para 55).

Besides two-dimensional arrays, three-dimensional arrays may also be formed. Three-dimensional cell arrays are commonly used in tissue engineering and biotechnology for in-vitro and in-vivo cell culturing. In general, a three-dimensional array is one which includes two or more layers separately applied to a substrate, with subsequent layers applied to the top surface of previous layers. The layers can, in one embodiment, fuse or otherwise combine following application or, alternatively, remain substantially separate and divided following application to the substrate. Three-dimensional arrays may be formed in a variety of ways in accordance with the present invention. For example, in one embodiment, three-dimensional arrays may be formed by printing multiple layers onto the substrate. (T. Boland at para 60).

The thickness of a printed layer (e.g., cell layer, support layer, etc.) may generally vary depending on the desired application. For example, in some embodiments, the thickness of a layer containing cells is from about 2 micrometers to about 3 millimeters, and in some embodiments, from about 20 micrometers to about 100 micrometers. Further, as indicated above, support compounds, such as gels, are often used to facilitate the survival of printed cells. The present inventors have discovered that the development of a cellular assembly may be increased when the thickness of the support layer(s) (e.g., between cells) is approximately the same as the size of the cells deposited adjacent to the support compound (T. Boland at para 61). When printing certain types of two-dimensional or three-dimensional arrays, it is sometimes desired that any subsequent cell growth is substantially limited to a predefined region. Thus, to inhibit cell growth outside of this predefined region, compounds may be printed or otherwise applied to the substrate that inhibit cell growth and thus form a boundary for the printed pattern. Some examples of suitable compounds for this purpose include, but are not limited to, agarose, poly(isopropyl N-polyacrylamide) gels, and so forth. In one embodiment, for instance, this “boundary technique” may be employed to form a multi-layered, three-dimensional tube of cells, such as blood vessels. For example, a cell suspension may be mixed with a first gel (“Gel A”) in one nozzle, while a second gel (“Gel B”) is loaded into another nozzle. Gel A induces cell attachment and growth, while Gel B inhibits cell growth. To form a tube, Gel A and the cell suspension are printed in a circular pattern with a diameter and width corresponding to the diameter and wall thickness of the tube, e.g., from about 3 to about 10 millimeters in diameter and from about 0.5 to about 3 millimeters in wall thickness. The inner and outer patterns are lined by Gel B defining the borders of the cell growth. For example, a syringe containing Gel A and “CHO” cells and a syringe containing Gel B may be connected to the nozzle. Gel B is printed first and allowed to cool for about 1 to 5 minutes. Gel A and CHO cells are then printed on the agarose substrate. This process may be repeated for each layer. (T. Boland at para 62).

The present invention particularly provides for the printing of tissues by the appropriate combination of cell and support material, or two or three or more different cell types typically found in a common tissue, preferably along with appropriate support compound or compounds, and optionally but preferably with one or more appropriate growth factors. Cells, support compounds, and growth factors may be printed from separate nozzles or through the same nozzle in a common composition, depending upon the particular tissue (or tissue substitute) being formed. Printing may be simultaneous, sequential, or any combination thereof. Some of the ingredients may be printed in the form of a first pattern (e.g., an erodable or degredable support material), and some of the ingredients may be printed in the form of a second pattern (e.g., cells in a pattern different from the support, or two different cell types in a different pattern). Again the particular combination and manner of printing will depend upon the particular tissue. Materials to be printed for specific tissues or tissue substitutes are described further below.

Skin. In representative embodiments, to produce epidermal-like skin tissue, the following are printed:
(a) at least one cell type, and preferably at least two or in some embodiments three or four different epidermal cell types (e.g., keratinocytes, melanocytes, Merkel cells, Langerhan cells, etc., and any combination thereof); and/or
(b) at least one support compound such as described above (e.g., collagen, elastin, keratin, etc., and any combination thereof); and/or
(c) at least one growth factor as described above (e.g., basic fibroblast growth factor (bFGF), Insulin-Like Growth Factor 1, epidermal growth factor (EGF), etc., and any combination thereof);

In some embodiments the epidermal cells, support compound and/or growth factors printed as described above (which form an “epidermal” type layer) are printed on, or on top of, a previously formed (e.g., printed or ink-jet printed) “dermal” type layer, the previously printed dermal layer layers comprising: (a) one, two, three or four different dermal cells (fibroblasts, adipocytes, mast cells, and/or macrophages), (b) at least one support compound as described above; and/or (c) at least one growth factor as described above.

Skin tissue produced by the process of the present invention is useful for implantation into or on a subject to, for example, treat burns, and other wounds such as incisions, lacerations, and crush injuries (e.g., postsurgical wounds, and posttraumatic wounds, venous leg ulcers, diabetic foot ulcers, etc.)

Bone. In particular embodiments, to produce bone tissues, the following are printed:
(a) at least one bone cell type, and preferably at least two or three different bone cell types (e.g., osteoblasts, osteoclasts, osteocytes, and any combination thereof, but in some embodiments at least osteoblasts and osteoclasts, and in some embodiments all three); and/or
(b) at least one support compound such as described above (e.g., collagen, hydroxyapatites, calicite, silica, ceramic, proteoglycans, glycoproteins, etc., and any combination thereof); and/or
(c) at least one growth factor (e.g., bone morphogenetic protein, transforming growth factor, fibroblast growth factors, platelet-derived growth factors, insulin-like growth factors, etc., and any combination thereof). Bone tissues produced by the processes described herein are useful for, among other things, implantation into a subject to treat bone fractures or defects, and/or promote bone healing.

Pancreatic. In representative embodiments, to produce pancreatic islet tissues, the following are printed:
(a) at least one, two, or three different pancreatic islet cell type (e.g., glucagon-synthesizing A (α) cells, insulin-producing B (β) cells, D (δ) cells, etc., and any combination thereof); and/or
(b) at least one support compound such as described above (e.g., collagen, proteoglycans, glycoproteins, elastin, etc., and any combination thereof); and/or
(c) at least one growth factor (e.g., insulin-Like Growth Factor II (IGF-II), gastrin, transforming growth factor-alpha (TGF alpha), vascular endothelial growth factor (VEGF), etc., and any combination thereof)

Pancreatic islet tissue produced by the processes described herein is useful for, among other things, implantation into a subject to treat diabetes (including type I and type II diabetes).

Nerve. In representative embodiments, to produce nerve tissue, the following are printed:
(a) at least one, two or three cells types, and preferably (i) a central or peripheral nerve cells (e.g., cortical neurons, hippocampal neurons, dopaminergic neurons, cholinergic neurons, adrenergic neurons, noradrenergic neurons, etc., including any combination thererof), and/or (ii) at least one glial cell type (e.g., neuroglia, astrocytes, oligodendrocytes, Schwann cells, etc., including any combination thereof) and (iii) any combination thereof (e.g., a combination of at least one nerve cell and at least one glial cell); and/or
(b) at least one support compound such as described above; (e.g., laminin, collagen type IV, fibronectin, etc., and any combination thereof); and/or
(c) at least one growth factor (e.g., NGF, brain-derived neurotrophic factor, insulin-like growth factor-I, fibroblast growth factor, etc., or any combination thereof); and any combination of the foregoing.

Nerve tissue produced by the processes described herein is useful, among other things, for implantation into a subject to treat nerve injury or degenrative diseases such as Parkinson's disease and Alzheimer's disease.

B. Stem cells.

In some embodiments stem cells are printed onto substrates by ink-jet printing. Stem cells may be printed alone (typically in combination with a support compound or compounds) or in combination with one or more additional cells (e.g., in a combination selected to produce a tissue as described above).

Stem cells (such as pluripotent or multipotent cells) are capable of differentiating into multiple different cell types or lines, including at least one of a hepatogenic-specific (or liver-specific) cell line, a myogenic (or muscle specific) cell line, an osteogenic (or bone specific) cell line, or an endothelial specific cell line. Useful cells for carrying out the invention include but are not limited to embryonic stem cells, parthenogenetic stem cells, amniotic fluid stem cells, and adipose-derived stem cells.

Embryonic stem cells useful for carrying out the present invention are known and described in, for example, U.S. Pat. No. 6,200,806 to Thomson and U.S. Pat. No. 5,843,780 to Thomson.

Adipose-derived stem cells are known and described in, for example, U.S. Pat. No. 6,777,231 to Katz et al.

Parthenogenetic stem cells useful for carrying out the present invention are known and described in, for example, J. Hipp et al., Parthenogenetic Stem Cells, in Myers, R. A. (Ed.): Meyers Encyclopedia of Molecular Cell Biology and Molecular Medicine, Vol. 10, pp. 71-84 (2d Ed. 2005) and K. Vrana et al., Non-human Primate Parthenogenetic Stem Cells, Proc. Natl. Acad. Sci. USA 100 Suppl 1: 11911-6 (2003).

Amniotic fluid stem cells (AFSCs) useful for carrying out the present invention are known and described in, for example, PCT Application WO 03/042405 to Atala and DeCoppi; In 't Anker, P. S., et al., Amniotic fluid as a novel source of mesenchymal stem cells for therapeutic transplantation. Blood, 2003. 102(4): p. 1548-9; Prusa, A. R., et al., Oct-4-expressing cells in human amniotic fluid: a new source for stem cell research? Hum Reprod, 2003. 18(7): p. 1489-93; Kaviani, A., et al., The amniotic fluid as a source of cells for fetal tissue engineering. J Pediatr Surg, 2001. 36(11): p. 1662-5; Prusa, A. R. and M. Hengstschlager, Amniotic fluid cells and human stem cell research: a new connection. Med Sci Monit, 2002. 8(11): p. RA253-7.

In general, AFSCs are cells, or progeny of cells, that are found in or collected primarily from mammalian amniotic fluid, but may also be collected from mammalian chorionic villus or mammalian placental tissue. The cells are preferably collected during the second trimester of gestation. In mice the cells are most preferably collected during days 11 and 12 of gestation. Preferably the mammalian source is of the same species as the mammalian subject being treated.

In general, the tissue or fluid can be withdrawn by amniocentesis, punch-biopsy, homogenizing the placenta or a portion thereof, or other tissue sampling techniques, in accordance with known techniques. From the sample, stem cells or pluripotent cells may be isolated with the use of a particular marker or selection antibody that specifically binds stem cells, in accordance with known techniques such as affinity binding and/or cell sorting. Particularly suitable is the c-Kit antibody, which specifically binds to the c-kit receptor protein. C-kit antibodies are known (see, e.g., U.S. Pat. Nos. 6,403,559, 6,001,803, and 5,545,533). Particularly preferred is the antibody c-Kit(E-1), a mouse monoclonal IgG that recognizes an epitope corresponding to amino acids 23-322 mapping near the human c-kit N-terminus, available from Santa Cruz Biotechnology, Inc., 2145 Delaware Avenue, Santa Cruz, Calif., USA 95060, under catalog number SC-17806).

AFSCs used to carry out the present invention are pluripotent. Hence, they differentiate, upon appropriate stimulation, into at least osteogenic, adipogenic, myogenic, neurogenic, hematopoitic, and endothelial cells. Appropriate stimulation, for example, may be as follows: Osteogenic induction: The cKit⁺ cells are cultured in DMEM low glucose with 10% FBS supplementing with 100 nM dexamethasone (Sigma-Aldrich), 10 mM beta-glycerophosphate (Sigma-Aldrich) and 0.05 mM ascorbic acid-2-phosphate (Wako Chemicals, Irving, Tex.); Adipogenic induction: To promote adipogenic differentiation, c-Kit⁺ cells are seeded at density of 3000 cells/cm² in DMEN low glucose medium with 10% FBS supplemented with 1 μM dexamethasone, 1 mM 3-isobutyl-1-methylxantine, 10 μg/ml insulin and 60 μM indomethacin (all from Sigma-Aldrich); Myogenic induction: c-Kit⁺ cells were plated into Matrigel-precoated dish (1 mg/ml, Collaborative Biomedical Products) and cultured in myogenic medium (DMEM low glucose supplemented with 10% horse serum, and 0.5% chick embryo extract from Gibco) followed by treatment of 5-azacytidine (10 μM, Sigma) added in myogenic medium for 24 h; Endothelial induction: c-Kit⁺ cells are plated into gelatin-precoated dish and cultured in endothelial basal medium-2 (EBM-2, Clonetics BioWittaker) supplemented with 10% FBS and 1% glutamine (Gibco). In preferred embodiments no feeder layer or leukaemia inhibitory factor (LIF) are required either for expansion or maintenance of AFSCs in the entire culture process.

AFSCs also have substantial proliferative potential. For example, they proliferate through at least 60 or 80 population doublings or more when grown in vitro. In preferred embodiments AFSCs used to carry out the invention proliferate through 100, 200 or 300 population doublings or more when grown in vitro. In vitro growth conditions for such determinations may be: (a) placing of the amniotic fluid or other crude cell-containing fraction from the mammalian source onto a 24 well Petri dish containing a culture medium [α-MEM (Gibco) containing 15% ES-FBS, 1% glutamine and 1% Pen/Strept from Gibco supplemented with 18% Chang B and 2% Chang C from Irvine Scientific], upon which the cells are grown to confluence, (b) dissociating the cells by 0.05% trypsin/EDTA (Gibco), (c) isolating an AFSC subpopulation based on expression of a cell marker c-Kit using mini-MACS (Mitenyl Biotec Inc.), (d) plating of cells onto a Petri dish at a density of 3−8×10³/cm², and (e) maintaining the cells in culture medium for more than the desired time or number of population doublings.

Preferably, the AFSCs are also characterized by the ability to be grown in vitro without the need for feeder cells (as described in PCT Application WO 03/042405 to Atala and DeCoppi. In preferred embodiments undifferentiated AFSCs stop proliferating when grown to confluence in vivo.

AFSCs used to carry out the present invention are preferably positive for alkaline phosphatase, preferably positive for Thy-1, and preferably positive for Oct4, all of which are known markers for embryonic stem cells, and all of which can be detected in accordance with known techniques. See, e.g., Rossant, J., Stem cells from the Mammalian blastocyst. Stem Cells, 2001. 19(6): p. 477-82; Prusa, A. R., et al., Oct-4-expressing cells in human amniotic fluid: a new source for stem cell research? Hum Reprod, 2003. 18(7): p. 1489-93.

In a particularly preferred embodiment, the AFSCs do not form a teratoma when undiferentiated AFSCs are grown in vivo. For example, undifferentiated AFSCs do not form a teratoma within one or two months after intraarterial injection into a 6-8 week old mouse at a dose of 5×10⁶ cells per mouse.

In preferred embodiments the amniotic fluid stem cells used to carry out the present invention express several markers characteristic of ES cells and/or various multipotent adult stem cells. These include the transcription factor Oct-4 (Pou5f1), SSEA-1 (Stage Specific Embryonic Antigen 1), Sca-1 (Ly-6A/E), CD90 (Thy-1), and CD44 (Hyaluronate receptor. Ly-24, P-1).

In preferred embodiments the amniotic fluid stem cells used to carry out the present invention do not express CD34 and CD105, markers of certain lineage restricted progenitors, nor the hematopoietic marker CD45.

In preferred embodiments the amniotic fluid stem cells used to carry out the present invention express low levels of major histocompatibility (MHC) Class I antigens and are negative for MHC Class II.

Differentiation of cells. “Differentiation” and “differentiating” as used herein include (a) treatment of the cells to induce differentiation and completion of differentiation of the cells in response to such treatment, both prior to printing on a substrate, (b) treatment of the cells to induce differentiation, then printing of the cells on a substrate, and then differentiation of the cells in response to such treatment after they have been printed, (c) printing of the cells, simultaneously or sequentially, with a differentiation factor(s) that induces differentiation after the cells have been printed, (d) contacting the cells after printing to differentiation factors or media, etc., and combinations of all of the foregoing. In some embodiments differentiation may be modulated or delayed by contacting an appropriate factor or factors to the cell in like manner as described above. In some embodiments appropriate differentiation factors are one or more of the growth factors described above. Differentiation and modulation of differentiation can be carried out in accordance with known techniques, as described in greater detail below, or as described in U.S. Pat. No. 6,589,728, or US Patent Application Publication Nos.: 2006006018 (endogenous repair factor production promoters); 20060013804 (modulation of stein cell differentiation by modulation of caspase-3 activity); 20050266553 (methods of regulating differentiation in stem cells); 20050227353 (methods of inducing differentiation of stem cells); 20050202428 (pluripotent stem cells); 20050153941 (cell differentiation inhibiting agent, cell culture method using the same, culture medium, and cultured cell line); 20050131212 (neural regeneration peptides and methods for their use in treatment of brain damage); 20040241856 (methods and compositions for modulating stem cells); 20040214319 (methods of regulating differentiation in stem cells); 20040161412 (cell-based VEGF delivery); 20040115810 (stem cell differentiation-inducing promoter); 20040053869 (stem cell differentiation); or variations of the above or below that will be apparent to those skilled in the art.

Pancreas. Differentiation of cells to pancreatic-like cells can be carried out in accordance with any of a variety of known techniques. For example, the cells can be contacted to, printed with, or cultured in a conditioning media such as described in US Patent Application 2002/0182728 (e.g., a medium that comprises Dulbecco's Minimal Essential Medium (DMEM) with high glucose and sodium pyruvate, bovine serum albumin, 2-mercaptoethanol, fetal calf serum (FCS), penicillin and streptomycin (Pen-Strep), and insulin, transferrin and selenium). In another example, the cells may be treated with a cAMP upregulating agent to induce differentiation as described in U.S. Pat. No. 6,610,535 to Lu. In still another example, the cells may be grown in a reprogramming media, such as described in US Patent Application 2003/0046722A1 to Collas to induce differentiation to a pancreatic cell type. In another embodiment, differentiation may be carried out using the 5 steps protocol describe by Lumelsky at al. Lumelsky, N., et al., Differentiation of embryonic stem cells to insulin-secreting structures similar to pancreatic islets. Science, 2001. 292(5520): p. 1389-94. In another embodiment, differentiation may be carried out using DMSO to induce pancreatic differentiation in vitro. She-Hoon Oh et al, Adult bone marrow-derived cells trans-differentiating into insulin producing cells for the treatment of type I diabetes. Lab Inv, 2004, 84: 607-617. In another embodiment, differentiation may be carried out using Nicotinamide to induce pancreatic differentiation in vitro. See, e.g., Otonkoski, T., et al, Nicotinamide is a potent inducer of endocrine differentiation in cultured human fetal pancreatic cells. J Clin Invest, 1993, 92(3): 1459-1466. In another embodiment, differentiation may be carried out using inhibitors of phosphoinositide 3-kinase (PI3K), such as LY294002, to induce pancreatic differentiation in vitro. See, e.g., Hori, Y., et al., Growth inhibitor promote differentiation of insulin producing tissue from embryonic stem cells. PNAS, 2002, 99:16105-16110. In another embodiment Exendin-4, a naturally occurring 39-amino acid peptide originally isolated from the salivary secretions of the Lizard Heloderma suspectum, can be used to induce pancreatic differentiation in vitro. Nielsen, L L., et al., Pharmacology of exenatide (synthetic exendin-4): a potential therapeutic for improved glycemic control of type 2 diabetes. Regul Pept. 2004 Feb 15;117(2):77-88. Review. In still another embodiment, anti-sonic hedgehog (Anti-Shh) and co-culturing with pancreatic rudiments can be used to induce pancreatic differentiation in vitro. Leon-Quinto, T., et al., In vitro direct differentiation of mouse embryonic stem cells into insulin producing cells. Diabetologia, 2004, 47:1442-1451. In one preferred embodiment the differenting step is carried out by transducing (sometimes also referred to as “engineering” or “transforming”) the cells with a vector, or introducing into the cells a vector, that contains a nucleic acid encoding a differentiation factor (such as Pdx1, Ngn3, Nkx6.1, Nkx2.1, Pax6, or Pax4) and expresses the differentiation factor in the cells, or by activating the expression of an endogeneous nucleic acid encoding a differentiation factor in the cells (e.g., engineering the cells to activate transcription of an endogeneous differentiation factor such as Pdx1, Ngn3, Nkx6.1, Nkx2.1, Pax6, or Pax4, such as by inserting a heterologous promoter in operative associated with an endogeneous differentiation factor, in accordance with known techniques. See, e.g., U.S. Pat. No. 5,618,698). Such exogeneous nucleic acids may be of any suitable source, typically mammalian, including but not limited to rodent (mouse, hamster, rat), dog, cat, primate (human, monkey), etc.

Osteogenic induction: Cells may be induced to form bone cells by any suitable technique, such as culturing in DMEN low glucose with 10% FBS supplementing with 100 nM dexamethasone (Sigma-Aldrich), 10 mM beta-glycerophosphate (Sigma-Aldrich) and 0.05 mM ascorbic acid-2-phosphate (Wako Chemicals, Irving, Tex.).

Adipogenic induction: Cells may be induced to promote adipogenic differentiation by any suitable technique, such as culturing in DMEN low glucose medium with 10% FBS supplemented with 1 μM dexamethasone, 1 mM 3-isobutyl-1-methylxantine, 10 μg/ml insulin and 60 μM indomethacin (all from Sigma-Aldrich);

Myogenic induction: Cells may be induced to promote myogenic induction by any suitable technique, such as culturing in myogenic medium (DMEM low glucose supplemented with 10% horse serum, and 0.5% chick embryo extract from Gibco) followed by treatment of 5-azacytidine (10 μM, Sigma) added in myogenic medium for 24 h.

Endothelial induction: Cells may be induced to promote endothelial induction by any suitable technique, such as culturing in endothelial basal medium-2 (EBM-2, Clonetics BioWittaker) supplemented with 10% FBS and 1% glutamine (Gibco).

The present invention is explained in greater detail in the following non-limiting examples.

### EXAMPLE 1

### Printing of Multiple Cell Types

Materials and Methods. Three distinct cell types were used in this study: human amniotic fluid-derived stem cells (hAFSC) transfected with lacZ, bladder smooth muscle cells (BSMC), and GFP labeled MS1 (mouse pancreatic islet endothelial cell line). Each cell type was grown separately, trypsinized, collected and resuspended in Type I collagen solution. Different mixtures of collagen and cells were loaded into different ink cartridges. Each cell-collagen mixture was printed layer-by-layer into the pre-designed target locations using a modified HP 550 printer. A solution containing NaOH was subsequently printed in order to neutralize the pH. The printed constructs were placed in the incubator for 3-5 hours. Once the collagen gel was set, 3-D viable multi-cellular constructs with a specific shape were formed. After 2 days of culture, the printed multi-cellular constructs were fixed and characterized using cell specific markers (α-actin, X-gal).

To examine the function of each cell type within the printed constructs, hAFSC cells were induced to differentiate into osteogenic lineage followed by evaluation of calcium production using Alizarin red staining Smooth muscle cell function was assessed by measuring the resting membrane potentials and K⁺ currents using a patch clamp system (Axopatch 200B).

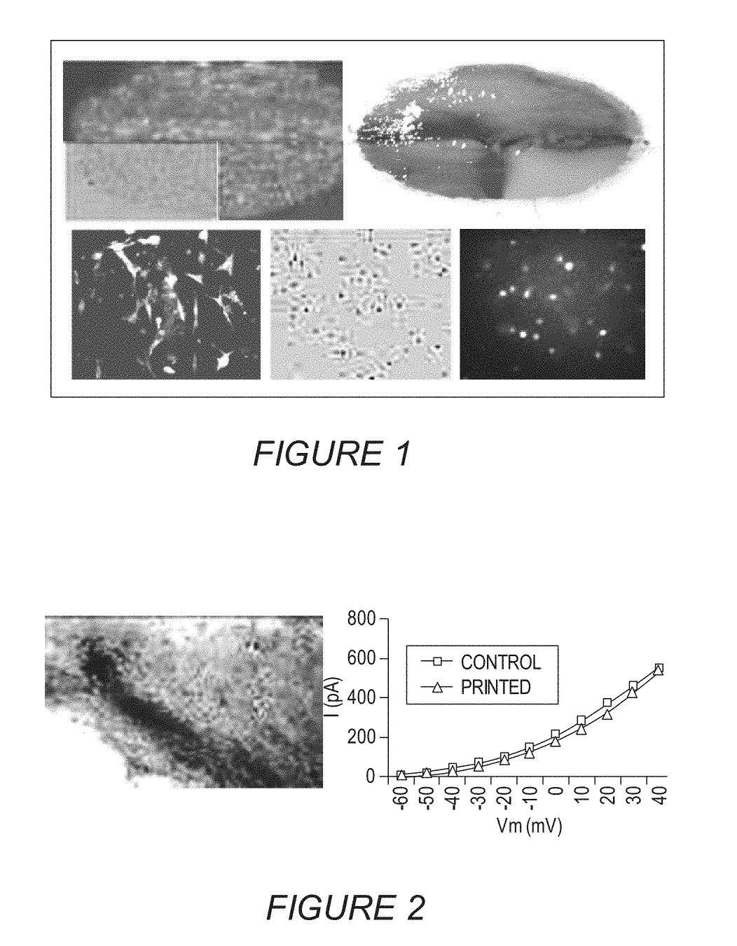

Results and Discussion. Fabrication of multi-cellular structures. All three printed cell types were confirmed by their corresponding cell identification methods, as shown in FIG. 1. The GFP labeled MS1 cells exhibited green fluorescence and smooth muscle cells emitted red under UV. The X-gal staining confirmed the lacZ transfected hAFSC cells in blue under bright field microscopy. All three cell types were present in an organized fashion within the printed construct. A 3-D collagen “pie” with different color dyes was shown in FIG. 1E, demonstrating the capability of the inkjet printers to print different biomaterials as well as multiple cell types.

Functional evaluation. Alizarin red staining showed the production of calcium in the osteogenic differentiation culture of hAFSC (FIG. 2a), which suggests that hAFSC in the collagen constructs retain their capability to differentiate into specific cell lineages under appropriate conditions. The whole cell patch clamp recording showed the average resting membrane potential of the printed BSMC (−58.5±5.8 mV), which is similar to normal non-printed smooth muscle cells (−54.7±7.5 mV). There was no significant difference on the K⁺ I-V relationship between the printed cells and the normal controls (FIG. 2b). These findings demonstrate that smooth muscle cells in the printed collagen constructs maintained their normal basic electrophysiological properties.

Conclusions. This example shows that viable three-dimensional heterogeneous constructs with multiple cell types can be generated by printing multiple cells and collagen gels layer-by-layer. These distinct cells are able to survive and proliferate within the 3-D constructs, and maintain normal basic cellular properties and function in their spatially registered regions. These findings demonstrate the possibility of building complex tissues that require multiple cell types and ECM materials by using the bio-printing technology.

### EXAMPLE 2

### In Vivo Generation of Tissues with Ink-Jet Printing

In this example we investigated whether the printed multi-cell derived tissue constructs could maintain their structural and spatial orientation in vivo. We examined whether these tissues are able to survive and mature into functional tissues when implanted in vivo.

Materials and Methods: Three-dimensional multi-cell constructs with a “pie” configuration were fabricated by simultaneously printing 3 different cell types [canine bladder smooth muscle cells (SMC), bovine aortal endothelial cells (EC), and human amniotic fluid-derived stem cells (AFSC)] into collagen/alginate gel. The cells were labeled with 3 different membrane bound tracers, which include [PKH67 (red), PKH26 (green), and CMHC (blue)], respectively, prior to printing. Individual cells were also printed separately for additional testing. The printed 3D constructs were subcutaneously implanted into athymic mice. AFSC-printed constructs were cultured in osteogenic medium for 1 week before implantation in order to induce differentiation into bone tissue. The implanted constructs were monitored by MRI and micro-CT scanner over time (up to 18 weeks). The retrieved engineered tissues were analyzed with confocal microscopy and immunohistochemical studies. To evaluate the function of the engineered muscle, electrophysiological properties were performed with voltage clamp experiments.

Results and Discussion.

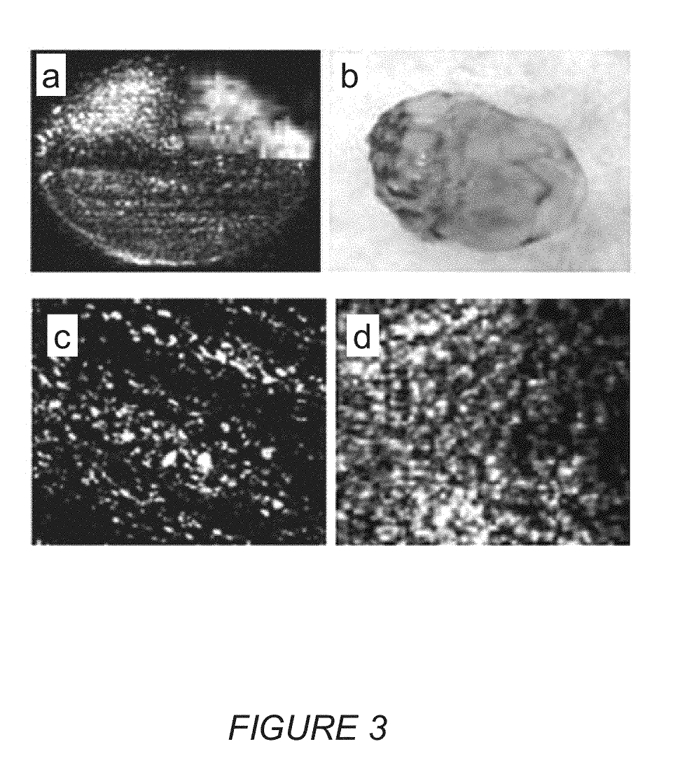

Printed multi-cell implants. A complete 3D “pie” shaped construct containing 3 different cell types (red dye tagged SMCs, green dye tagged ECs, and blue dye tagged AFSCs) was successfully fabricated by the inkjet method (FIG. 3). The tissue structure of the 3D “pie” was maintained 2 weeks after implantation (FIG. 3b). The membrane bound tracers confirmed that the printed cells remained viable in their pre-determined locations (Green dye stained ECs and red dye stained SMCs; (FIG. 3c, d).

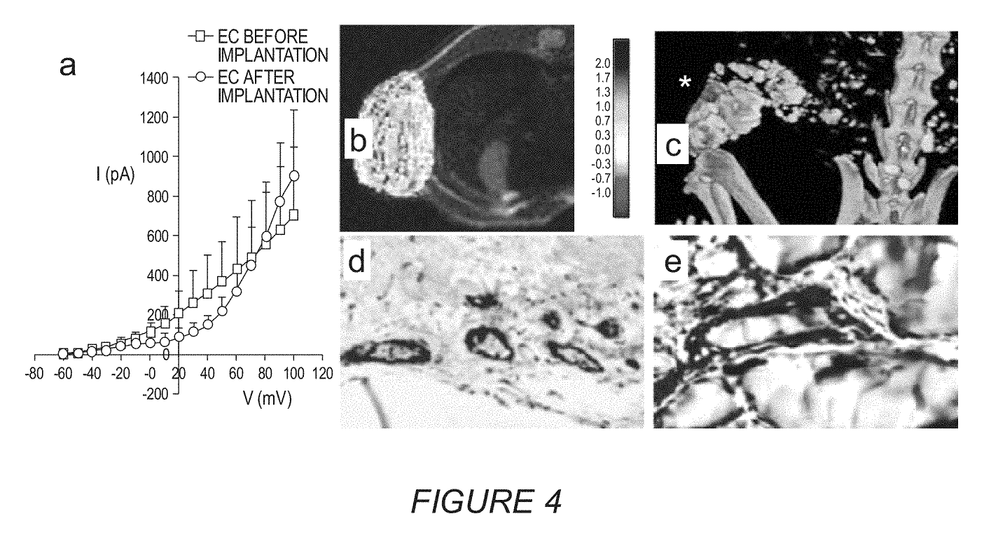

In vivo functional evaluation. The voltage clamp recording showed that the printed SMCs exhibited similar patterns in the mean current voltage (I-V) relationships before and after implantation (FIG. 4a), which suggests that the SMCs are able to maintain normal basic electrophysiological characteristics in vivo. Vascularization of the EC-printed implants was evaluated by MRI scanning 8 weeks post-implantation. After the gadolinium (Gd) contrast agent was injected intravenously into the animal, contrast enhancement was visualized within the implants, which indicates the presence of vascular network within the implanted tissues. FIG. 4b shows intensified MRI signals, and the degree of contrast enhancements is denoted in different colors in the implants. The formation of vascularization was reconfirmed by the presence of blood vessels, which were positively expressed with the endothelial cell-specific marker: vWF (FIG. 4d). These data suggest that EC-printed implants are able to form functional vasculature. Micro CT scanning showed that bone-like hard tissues were formed within the AFSC-printed constructs 18 weeks post-implantation (FIG. 4c) Immuonohistochemical analysis showed that the differentiated AFSCS within the implant expressed a typical bone cell marker, osteocalcin (FIG. 3d). These data suggest that the printed stem cells within the constructs retain their ability to differentiate into specific cell lineages and enhance formation of relevant tissues under specific conditions.

Conclusions. This example shows that multi-cellular constructs, generated by the inkjet method, are able to maintain their structural and spatial orientation in vivo. The printed cells are able to retain their cellular characteristics and tissue function. The inkjet printing technology may become a standard method of engineering functional tissues for clinical applications.

The foregoing is illustrative of the present invention, and is not to be construed as limiting thereof. The invention is defined by the following claims, with equivalents of the claims to be included therein.

1. In a method of forming an array of viable cells by ink-jet printing a cellular composition containing said cells on a substrate, the improvement comprising:
printing at least two different types of viable mammalian cells on said substrate, said at least two different types of viable mammalian cells selected to together form a tissue.

2. The method of claim 1, wherein at least three different viable mammalian cells types are printed on said substrate, the cells selected to together form a tissue.

3. The method of claim 1, wherein at least one of said viable mammalian cell types is a stem cell.

4. The method of claim 1, further comprising printing at least one support compound on said substrate, said support compound selected to form a tissue together with said cells.

5. The method of claim 1, wherein said tissue is selected from the group consisting of nerve, skin, pancreatic islet, and bone tissue.

6. The method of claim 1, wherein said tissue is skin tissue.

7. The method of claim 1, wherein said tissue is bone tissue.

8. The method of claim 1, wherein said tissue is pancreatic islet tissue.

9. The method of claim 1, wherein said tissue is nerve tissue.

10. In a method of forming an array of viable cells by ink-jet printing a cellular composition containing said cells on a substrate, the improvement comprising:
printing viable stem cells on said substrate.

11. The method of claim 10, wherein said stem cells are amniotic fluid stem cells.

12. The method of claim 1, further comprising the step of implanting said array in vivo in a subject in need thereof.

13. The method of claim 12, further comprising maintaining said array in vivo in said subject for at least one month, during which all cell types in said array maintain their structural and spatial orientation in vivo.

14. The method of claim 12, further comprising maintaining said array in vivo in said subject for at least two months, during which all cell types in said array maintain their structural and spatial orientation in vivo and retain their cellular characteristics and tissue function.

15. In a method of forming an array of viable cells by ink jet printing a cellular composition containing said cells on a substrate, the improvement comprising:
printing viable cancer cells on said substrate.

16. The method of claim 15, wherein said cancer cells are selected from the group consisting of leukemia, lymphoma, breast, lung, colon, prostate, ovarian, skin, melanoma, and brain cancer cells.
